(19) United States
(12) Patent Application Publication
(10) Pub. No.: US 2006/0100190 A1
(43) Pub. Date: May 11, 2006

(54) Motexafin lutetium phototherapy with low fluences for treating vascular inflammation

(75) Inventors: Wai-Fung Cheong, Los Altos, CA (US); Yi-Ping Sun, San Jose, CA (US); Richard Miller, Portola Valley, CA (US)

Correspondence Address: WILSON SONSINI GOODRICH & ROSATI, 650 PAGE MILL ROAD, PALO ALTO, CA, 94304-1050, US

(73) Assignee: Pharmacyclics, Inc.

(21) Appl. No.: 11/267,104

(22) Filed: Nov. 4, 2005

Related U.S. Application Data
(60) Provisional application No. 60/625,477, filed on Nov. 5, 2004.

(51) Int. Cl. A61K 31/555; A61N 1/30

(52) U.S. Cl. 514/185; 604/20

(57) ABSTRACT

Described herein are compositions, methods, therapies, strategies, techniques and processes for treating vascular inflammation in blood vessels by administering a photo-activatable compound and low light fluence in the range of about 10 to about 400 Joules/cm of illuminated length in the general vicinity of the inflammation.

### CROSS-REFERENCE

This application claims the benefit of U.S. Provisional Application No. 60/625,477, filed Nov. 5, 2004, which is incorporated herein by reference in its entirety.

### FIELD OF THE INVENTION

The methods, compositions, strategies and techniques described herein relate to treating regional inflammation in blood vessels by administering a photo-activatable compound and low light fluence.

### BACKGROUND OF THE INVENTION

Atherosclerosis is a chronic inflammatory disease characterized by diffuse inflammation of the blood vessels, increased vascular macrophages (Mac) and vulnerable plaque that are implicated in acute coronary syndrome and sudden death. A variety of agents and methods are available to tackle the disease. An effective treatment remains elusive. It has been reported that Motexafin Lutetium (MLu, Antrin®), a photoactive, synthetic expanded porphyrin, selectively accumulates in atheroma and destroys Mac by inducing apoptosis following phototherapy (PT).

### SUMMARY OF THE INVENTION

Described herein are methods, compositions, treatment strategies, processes and techniques for (a) regional treatment of vascular inflammation, including focal inflammation and diffuse inflammation; (b) treating inflammation in blood vessels, including native coronary arteries with or without vein or arterial grafts; (c) treating blood vessels at risk for plaque rupture, including blood vessels with focal inflammation or diffuse inflammation; (d) treating vulnerable plaque in blood vessels; (e) treating blood vessels containing diffuse involvement by atheromatous plaque disease, including segments of a blood vessel(s) at high risk for plaque rupture; (f) treating non-continuous inflammatory conditions involving at least one blood vessel; (g) treating inflammation involving multiple blood vessels; (h) treating non-continuous inflammation in multiple blood vessels; (i) treating undetected inflammation near detectable inflammation within a blood vessel; (j) reducing macrophage levels in a blood vessel, including macrophages that are present at a site of inflammation in a blood vessel; (k) decreasing plaque burden that contributes to inflammation in blood vessels, including diffuse inflammation; (l) promoting favorable remodeling (e.g., collagen stabilization) in blood vessels afflicted with inflammation, including diffuse inflammation; and (m) stabilizing plaque in blood vessels at risk for plaque rupture, including blood vessels with diffuse inflammation. Any of the aforementioned methods, compositions, treatment strategies, processes and techniques include aspects wherein (1) the site of inflammation includes vulnerable plaque; (2) the blood vessel is treated by administering to a host (including a human patient) of such treatment a photo-activatable compound, including a texaphyrin compound; (3) a photo-activatable compound accumulates at or near the disease site(s) in the blood vessel (including inflammation, vulnerable plaque, atheroma), including intracellular accumulation, extracellular accumulation, and a combination thereof; (4) a photo-activatable compound selectively accumulates at or near the disease site(s) in the blood vessel (including inflammation vulnerable plaque, atheroma); (5) the site of inflammation includes atheroma; (6) administration of a photo-activatable compound is followed after a period of time by providing to the host (including a human patient) light suitable to activate the photo-activatable compound; (7) light suitable to activate a photo-activatable compound is delivered endovascularly; (8) light suitable to activate a photo-activatable compound is delivered at low fluences, including in the range of from about 10 joules/cm to about 400 joules/cm (9) light suitable to activate a photo-activatable compound is delivered to inflamed segments of the blood vessels containing the inflammation; (10) activation of a photo-activatable compound initiates processes that result in the apoptosis of cells; (11) a photo-activatable compound, following light activation, induces apoptosis in cells; (12) apoptosis, induced by light activation of a photo-activatable compound occurs selectively at sites of inflammation, including sites of atheroma; (13) a paramagnetic compound is administered to enhance visualization of inflammation, vulnerable plaque, atheroma, and/or atheromatous plaque disease; (14) the disease site(s) in the blood vessel (including inflammation vulnerable plaque, atheroma) is treated by administering a texaphyrin compound, including a compound of Formula (I); (15) the disease site(s) in the blood vessel (including inflammation vulnerable plaque, atheroma) is treated by administering a combination of texaphyrin compounds, including a compound of Formula (I), M=Lu(III) and Gd(III); (16) the adventitial temperature of the illuminated blood vessel does not increase by more than about 2° C.; (17) the adventitial temperature of the illuminated blood vessel does not increase by more than about 4° C.; (18) light suitable to activate a photo-activatable compound is delivered using an optical fiber; (19) light suitable to activate a photo-activatable compound is delivered using a fiber optic with a distal light diffusing tip (i.e., diffuser); (20) light suitable to activate a photo-activatable compound is delivered using a bare fiber optic diffuser or a fiber optic diffuser placed inside a sheath (e.g., balloon catheter); (21) light suitable to activate a photo-activatable compound is delivered using an LED array; (22) light suitable to activate a photo-activatable compound is delivered an LEP tip; (23) light suitable to activate a photo-activatable compound is delivered using a centering catheter; (24) a photo-activatable compound is delivered systemically; (25) a photo-activatable compound is delivered intravenously; (26) a photo-activatable compound is delivered by means of local administration; (27) light suitable to activate a photo-activatable compound is delivered near the desired site in the blood vessel; (28) the blood vessel is in a human patient; (29) the segment of blood vessel requiring treatment is less than 1 cm in length; (30) the segment of blood vessel requiring treatment is more than 1 cm in length; (31) the segment of blood vessel requiring treatment is more than 10 cm in length; (32) the segment of blood vessel requiring treatment is more than 25 cm in length; (33) the segment of blood vessel requiring treatment is a long segment; and (34) and any and all combinations of the foregoing that are not logically mutually exclusive.

In one aspect are pharmaceutical compositions for the treatment of a disease site(s) in a blood vessel in a human patient (including inflammation vulnerable plaque, atheroma) comprising a compound having the structure of Formula (I): 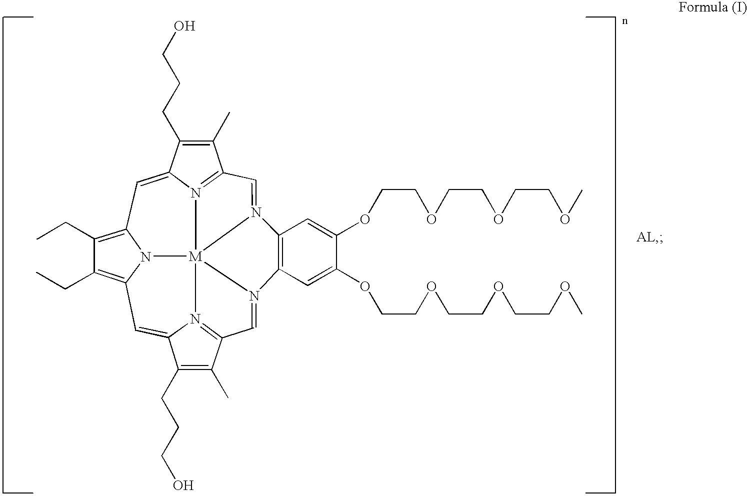
wherein M represents a diamagnetic metal cation selected from Lu(III), Y(III), Cd(II), In(III), and Zn(II); n is 1 or 2; each AL is independently selected from the group consisting of OH⁻, AcO⁻, Cl⁻, Br⁻, I⁻, F⁻, H₂PO₄⁻, ClO⁻, ClO₂⁻, ClO₃⁻, ClO₄⁻, HCO₃⁻, HSO₄⁻, NO₃⁻, N₃⁻, CN⁻, SCN⁻, and OCN⁻. In one embodiment, M is Lu(III). In one embodiment, n is 2 and each AL is AcO⁻. In a further or alternate embodiment, each AL is selected from the group consisting of sugar derivatives, cholesterol derivatives, PEG acids, organic acids, organosulfates, organophosphates, phosphates or inorganic ligands. In a further or alternate embodiment, AL is derived from an acid selected from the group consisting of gluconic acid, glucoronic acid, cholic acid, deoxycholic acid, methylphosphonic acid, phenylphosphonic acid, phosphoric acid, formic acid, propionic acid, butyric acid, pentanoic acid, 3,6,9-trioxodecanoic acid, 3,6-dioxoheptanoic acid, 2,5-dioxoheptanoic acid, methylvaleric acid, glycolic acid, pyruvic acid, oxalic acid, malic acid, malonic acid, succinic acid, maleic acid, fumaric acid, tartaric acid, citric acid, methanesulfonic acid, ethanesulfonic acid, benzoic acid, salicylic acid, 3-fluorobenzoic acid, 4-aminobenzoic acid, cinnamic acid, mandelic acid, and p-toluene-sulfonic acid. The compound of Formula (I) belongs to the class of compounds known as metallated texaphyrins. In further embodiments of such compositions, at least about 98.7%, 99%, 99.3% or 99.5% of the metallated texaphyrins in the composition have the structure of Formula (I) (and the same molecular weight, excluding isotopic variation), i.e. both polyethylene glycol chain lengths on the aromatic moiety have the same chain length (including the same “x” value per the generic formula). In further or alternative embodiments, the pharmaceutical composition comprises (i) water; (ii) acetic acid; (iii) mannitol, and combinations thereof. In further or alternative embodiments, the pharmaceutical composition is stored under at least about 90% nitrogen.

The photo-activatable agents disclosed herein can also be used diagnostically (e.g. fluorescence or optical absorbance imaging to detect the presence of a disease). Alternatively, if other detection means, such as MRI, are used for diagnostic purposes, then the M in the compound of Formula (I) can be a paramagnetic metal ion, including Gd(III). The use of M=Gd(III) forms of a compound of Formula (I) for diagnostic detection of vascular inflammation (including diffuse inflammation) is considered within the scope of the compositions, methods, strategies, processes and techniques described herein. For example, administration of a compound of Formula (I) [Gd(III)=M] to visualize sites of vascular inflammation and administration of a compound of Formula (I) [Lu(III)=M] to treat vascular inflammation is one approach described herein for the treatment of vascular inflammation. Sequential MR imaging of the target tissue (plaque) is also possible. MR images collected as a compound of Formula (I) [Gd(III)=M] washes in and out of the different cells and tissue compartments allows formation of a composite molecular picture of the tissue or lesion. The dynamics occur at different rates, depending on the structure and formulation of the compound of Formula (I) [Gd(III)=M], and the imaging sequence used (e.g., T1-weighted, T2-weighted, Proton Density Weighted, FSE, TR, TE). For example, lipids appear hyperintense under T1W protocol but hypointense under T2W protocol.

Also encompassed within the methods, compositions, techniques and strategies described herein are combinations of diagnostic applications (e.g., visualizing vascular inflammation using a compound of Formula (I) [Gd(III)=M]) and therapeutic applications (e.g., treating vascular inflammation using a compound of Formula (I) [Lu(III)=M]). The diagnostic steps can occur prior to the therapeutic steps, simultaneous with the therapeutic step (e.g., administering a formulation comprising both a compound of Formula (I) [Gd(III)=M] and a compound of Formula (I) [Lu(III)=M]), after the therapeutic steps (to monitor the progress of the therapeutic steps), or a combination of the foregoing.

Certain Definitions

Antrin, as that term is used herein, refers to a compound of Formula (I) wherein M is Lu(III), n is 2 and AL is acetate. Antrin is also known as MLu and motexafin lutetium.

Atheroma as that term is used herein refers to the arterial chronic inflammatory response to vascular endothelial injury (a complex lesion). It includes an accumulation of lipid, increased inflammatory cells (e.g., macrophages, lymphocytes), proliferated smooth muscle cells, and other extra cellular matrix on the inner lining of the artery or blood vessel.

Blood vessels covers all vasculature, including native coronary arteries, arterial grafts, vein grafts, peripheral arteries.

Photo-activatable or light activatable compound as those terms are used herein refers to a molecule that is activated when illuminated with light of a specific wavelength or spectral bandwidth, to produce an effect.

Light fluence as that term is used herein refers to the amount of light energy (in units of Joules) per unit length of source illumination (in centimeter). It is also referred to as light energy.

Illuminated length as that term is used herein refers to the length of vascular segment receiving light, which is typically referenced to a multiple of the active diffuser length.

Macrophage as that term is used herein refers to a type of white blood cell that is found all over our body, that surrounds and kills microorganisms (e.g., debris, foreign substances/cells), removes dead cells, and stimulates the action of other immune system cells, thus protecting the body .

General vicinity of the atheroma as that term is used herein refers to includes the atheroma and areas surrounding it.

Intravascular administration as that term is used herein refers to is a mode of delivery within the lumen of a blood vessel.

Light delivery device as that term is used herein refers to is a passive device which transports light from an external source to the site of delivery or an active device which generates light at the site of delivery.

Optical Fiber as that term is used herein refers to is a thin filament of drawn or extruded glass or plastic having a central core and a cladding of lower refractive index to promote internal reflection.

Fiber optic diffuser as that term is used herein refers to a specially designed optical fiber that terminates at one end in a fiber optic connector to a light source, and at the other end in a special tip that emits light in a pre-determined geometry, example, a cylindrical diffuser emitting 360° of light circumferentially, or a spherical diffuser emitting over a 4° solid angle

Bare Fiber as that term is used herein refers to a light deliver device being used whereby the active diffuser or light emission portion is exposed (not covered with a sheath or catheter) during delivery of light to the target tissue.

Light emitting diode (LED) as that term is used herein refers to a device designed to use stimulated emission to form a coherent light output.

Light emitting polymer (LEP) as that term is used herein refers to a (also known as polymer LED) materials where the polymer is the semiconductor material used in the LED. Polymers are chemical substances that consist of large molecules that are, themselves, made from many smaller and simpler molecules: proteins and DNA are examples of naturally occurring polymers; many others, such as nylon, are artificially created. Hence LEP devices are flexible.

Centering Catheter as that term is used herein refers to a catheter that when positioned, is centered within the vessel lumen along the long axis of the vessel.

Adventitial temperature as that term is used herein refers to the temperature measured at the adventitial surface of a blood vessel that is receiving intravascular illumination.

Systemic administration as that term is used herein refers to an intravenous injection or delivery of a compound such that the compound circulates in the vasculature (e.g., veins, venules, arteries, arterioles, capillary bed) of a body

Local administration as that term is used herein refers to injection or delivery of a compound in the vicinity of the target.

Texaphyrins as that term is used herein refers to an aromatic pentadentate macrocyclic expanded porphyrins, also described as an aromatic benzannulene containing both 18π- and 22π-electron delocalization pathways. Texaphyrins and water-soluble texaphyrins, methods of preparation and various uses and the like have been described, for example, in U.S. Pat. Nos. 4,935,498, 5,162,509, 5,252,720, 5,256,399, 5,272,142, 5,292,414, 5,369,101, 5,432,171, 5,439,570, 5,451,576, 5,457,183, 5,475,104, 5,504,205, 5,525,325, 5,559,207, 5,565,552, 5,567,687, 5,569,759, 5,580,543, 5,583,220, 5,587,371, 5,587,463, 5,591,422, 5,594,136, 5,595,726, 5,599,923, 5,599,928, 5,601,802, 5,607,924, 5,622,946, 5,714,328, 5,733,903, 5,744,302, 5,756,726, 5,763,172, 5,775,339, 5,776,925, 5,798,491, 5,801,229, 5,808,059, 5,817,017, 5,837,866, 5,886,173, 5,888,997, 5,955,586, 5,969,111, 5,994,935, 6,022,526, 6,022,959, 6,069,140, 6,072,038, 6,096,030, 6,270,749, 6,375,930, 6,638,924, 6,657,058, 6,825,186, 6,919,327; in PCT publications WO 90/10633, 94/29316, 95/10307, 95/21845, 96/09315, 96/40253, 96/38461, 97/26915, 97/35617, 97/46262, 98/07733, 98/25648, 99/09411, 99/15236, 99/62551, 00/01413, 00/01414; in allowed U.S. patent application Ser. No. 09/325,890; and in pending U.S. patent application Ser. Nos. 08/935,412, 08/975,090, 09/329,720, 09/431,298, 09/699,027, 60/229,247, 60/229,255, 60/229,366 and 60/249,523, each of which are herein incorporated by reference in their entirety.

### BRIEF DESCRIPTION OF THE DRAWINGS

FIG. 1 depicts treated segments of the rabbit aortic tree. In each rabbit, both T1 and T2 segments were about 30 mm in length with another 30 mm of buffer in between. Both T1 and T2 received the same fluence (J/cmf) at different fluence rates (mW/cmf) because T1 illumination period was shorter at 400 seconds compared to 900 seconds for T2.

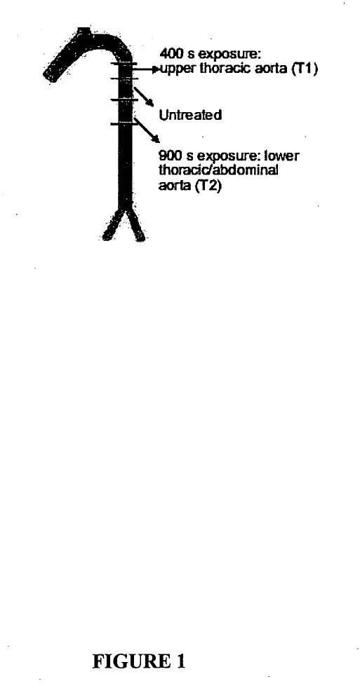

FIG. 2 shows that low light fluences affected a decrease in plaque burden based on intima/media ratios, normalized to control. Plaque reduction was statistically significant in T2 (900s) segments for all tested fluences vs. control (p<0.05) and only in T1 groups at lower fluences of 30-75 J/cmf vs. control (p<0.02). Fluence rate is thus as important as fluence in determining treatment success in regressing plaque.

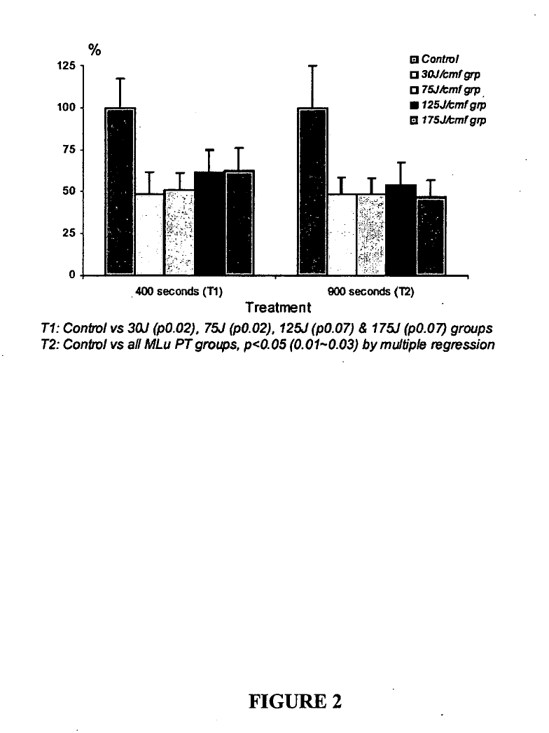

FIG. 3 shows a statistical significant reduction in macrophage burden obtained using all tested fluence groups (30-175 J/cmf) vs. control, p<0.05, in both T1 and T2 treated segments.

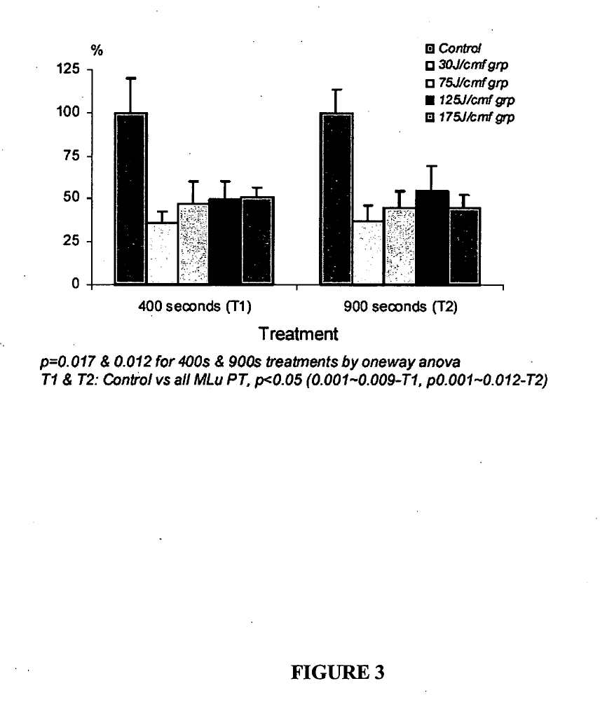

FIG. 4 depicts representative histology of smooth muscle cell (SMC) content in control (non-phototherapy) and treated (30 J/cmf, 400 s) vessel segments. SMC content did not appear to decrease following Antrin phototherapy based on myosin-stained areas colored in brown. Planimetry of the treated sections showed slight increase in SMC content (myosin positive, brown areas) with 50.9±9.7% vs. 43.1±3.8% of the initima (control).

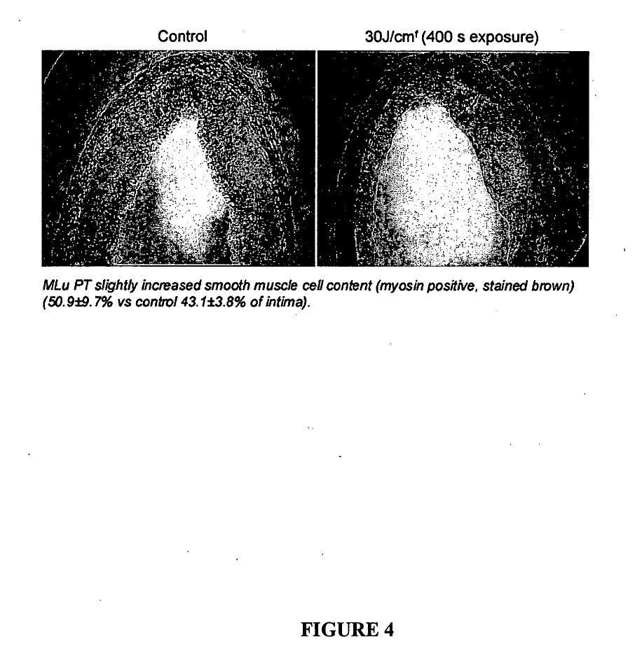

FIG. 5 depicts bright-field and polarization microscopic images of control and treated (30 J/cmf, 400 s) segments stained with picrosirius red for collagen. The loosely packed collagen indicated that new collagen infiltrated atheromas of control rabbits; new collagen appears as green-yellow under polarization. In treated rabbits, the lesions showed denser packing of matured collagen, suggesting potential favorable collagen remodeling by MLu PT, and hence the potential to stabilize plaque.

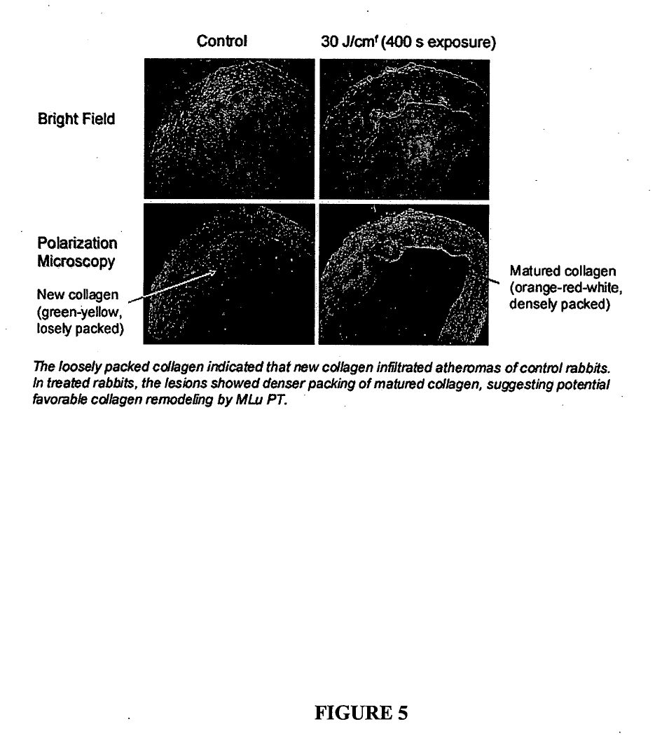

FIG. 6 depicts microscopic images of a vascular segment from a control rabbit in which the segment was stained with Oil Red O or RAM 11. The images show that neutral lipids accumulate or colocalize in areas with significant populations of foamy macrophages.

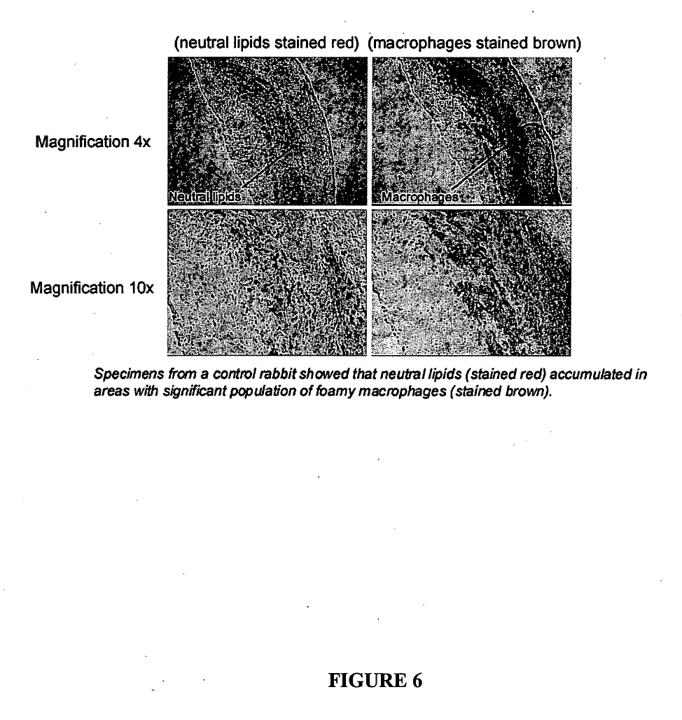

### DETAILED DESCRIPTION OF THE INVENTION

It has been surprisingly found that fat-fed New Zealand rabbits treated with MLu followed by phototherapy (PT, at 732 nm) at low light fluence (30-100 J/cmf) showed significant reduction in diffuse inflammation (based on decrease in macrophage burden) in blood vessels. Fat-fed Watanabe Heritable Hyperlipidemic (WHHL) rabbits subjected to a wider dose of light (30-175 J/cmf) delivered over 400 s and 900 s similarly showed macrophage depletion in their plaques. The methods, compositions, strategies and techniques described herein include processes to treat diffuse inflammation in blood vessels by administering a photo-activatable compound and low light fluence in the range of in the range of about 10 to about 400 Joules/cm of illuminated length in the general vicinity of the inflammation.

The methods, compositions, strategies and techniques described herein include therapies for diffuse inflammation involving blood vessels at risk for plaque rupture comprising administering to a host (including a human patient) in need of such treatment a photo-activatable compound and light delivered endovascularly at a fluence in the range of about 10 to about 400 Joules/cm to long segments of the blood vessels. One embodiment provides a treatment wherein the light activatable compound has a structure of Formula (I) 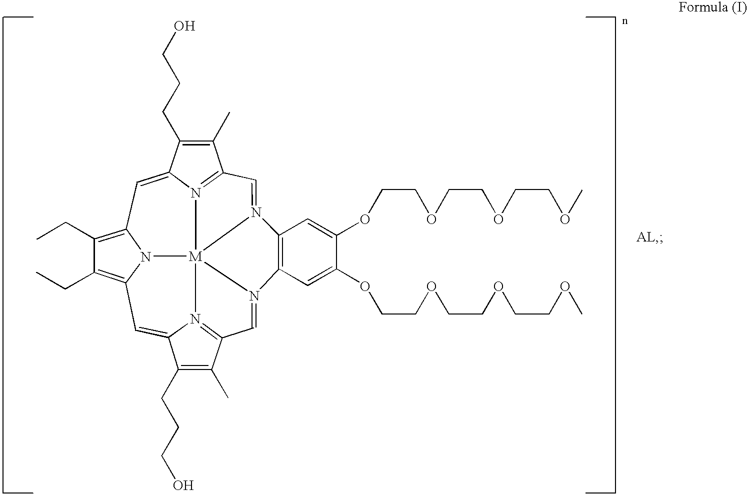
wherein M represents a diamagnetic metal cation selected from Lu(III), Y(III), Cd(II), In(III), and Zn(II). A preferred embodiment provides a process wherein the compound of Formula (I) is motexafin lutetium (MLu, M represents Lu (III)); and wherein the host (including a human patient) is administered: a) from about 0.5 mg/Kg to about 4.0 mg/Kg of MLu; and (b) light fluence in the range of about 20 to about 100 joules/cm of the long segments of the blood vessels.

Another aspect of the methods, compositions, strategies and techniques described herein are therapies for vulnerable plaque involving segments (including segments over 1 cm in length, segments over 5 cm in length, segments over 10 cm in length, and segments over 25 cm in length; further including continuous or discontinuous segments) of a coronary artery, arterial graft or vein graft, wherein the vulnerable plaque is composed of inflammatory cells and lipids, in a host (including a human patient) needing such treatment, such therapies comprising administering to the host (including a human patient) a photo-activatable compound and administering light endovascularly at a fluence in the range of about 10 to about 400 joules/cm. In one embodiment of this aspect are treatments wherein: a) the light activatable compound has a structure of Formula (I): 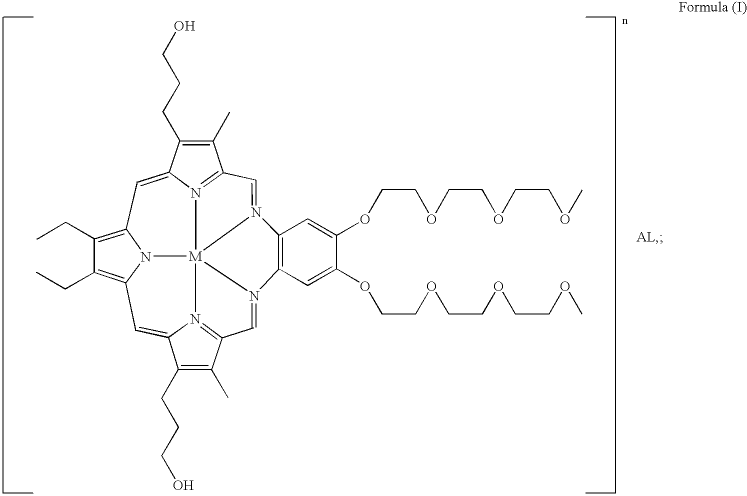
wherein M represents a diamagnetic metal cation selected from Lu(III), Y(III), Cd(II), In(III), and Zn(II). A preferred embodiment provides a process wherein the compound of Formula (I) is motexafin lutetium (MLu, M represents Lu (III)) and wherein the host (including a human patient) is administered: a) from about 0.5 mg/Kg to about 4.0 mg/Kg of MLu; and (b) light fluence in the range of about 20 to about 100 joules/cm.

Yet another aspect of the methods, compositions, strategies and techniques described herein are therapies for treating a long segment of a blood vessel containing diffuse involvement by atheromatous plaque disease which are at high risk for plaque rupture, the therapies comprising administering to a host (including a human patient) in need of such a treatment a photo-activatable compound and light delivered endovascularly at fluence in the range of from about 10 joules/cm to about 400 joules/cm along a long segment of the blood vessel without adversely affecting the blood vessel tissue not at risk of plaque rupture. In one embodiment of such an aspect are processes wherein: a) the light activatable compound has a structure of Formula (I) 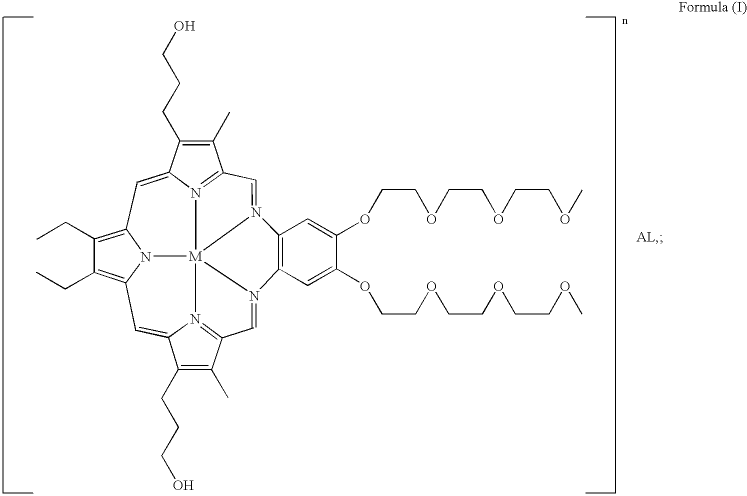
wherein M represents a diamagnetic metal cation selected from Lu(III), Y(III), Cd(II), In(III), and Zn(II). A preferred embodiment provides a process wherein the compound of Formula (I) is motexafin lutetium (MLu, M represents Lu (III)) and wherein the host (including a human patient) is administered: a) from about 0.5 mg/Kg to about 4.0 mg/Kg of MLu; and (b) light fluence in the range of about 20 to about 100 joules/cm.

Yet another aspect of the methods, compositions, strategies and techniques described herein are therapies for non-continuous inflammatory condition involving a blood vessel, the therapies comprising administering to a host (including a human patient) in need of such therapies a photo-activatable compound and light delivered endovascularly at fluence in the range of from about 10 joules/cm to about 400 joules/cm along a long segment of the blood vessel containing the non-continuous inflammation without adversely affecting the intervening non-inflamed regions of the blood vessel. In one embodiment of such an aspect are processes wherein: a) the light activatable compound has a structure of Formula (I): 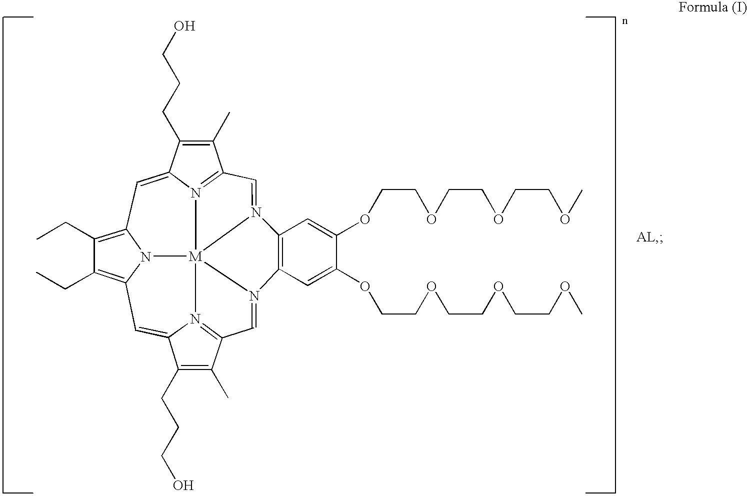
wherein M represents a diamagnetic metal cation selected from Lu(III), Y(III), Cd(II), In(III), and Zn(II). A preferred embodiment provides a process wherein the compound of Formula (I) is motexafin lutetium (MLu, M represents Lu (III)) and wherein the host (including a human patient) is administered: a) from about 0.5 mg/Kg to about 4.0 mg/Kg of MLu; and (b) light fluence in the range of about 20 to about 100 joules/cm.

Yet another aspect of the methods, compositions, strategies and techniques described herein are therapies for inflammation involving multiple blood vessels, the therapies comprising administering to a host (including a human patient) in need of such therapies a photo-activatable compound and light delivered endovascularly at fluence in the range of from about 10 joules/cm to about 400 joules/cm to inflamed segments of each of the multiple blood vessels containing the inflammation. In one embodiment of such an aspect are processes wherein: a) the light activatable compound has a structure of Formula (I): 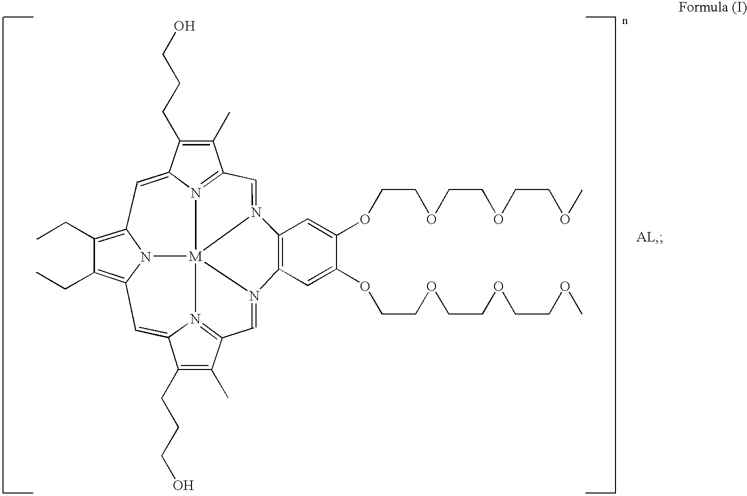
wherein M represents a diamagnetic metal cation selected from Lu(III), Y(II), Cd(II), In(III), and Zn(II). A preferred embodiment provides a process wherein the compound of Formula (I) is motexafin lutetium (MLu, M represents Lu (III)) and wherein the host (including a human patient) is administered: a) from about 0.5 mg/Kg to about 4.0 mg/Kg of MLu; and (b) light fluence in the range of about 20 to about 100 joules/cm.

Yet another aspect of the methods, compositions, strategies and techniques described herein are therapies for non-continuous inflammation in multiple blood vessels, the therapies comprising administering to a host (including a human patient) in need of such therapies a photo-activatable compound and light delivered endovascularly at fluence in the range of from about 10 joules/cm to about 400 joules/cm along the entire inflamed segments of the multiple segments of the blood vessels containing the non-continuous inflammation. In one embodiment of this aspect are processes wherein: a) the light activatable compound has a structure of Formula (I): 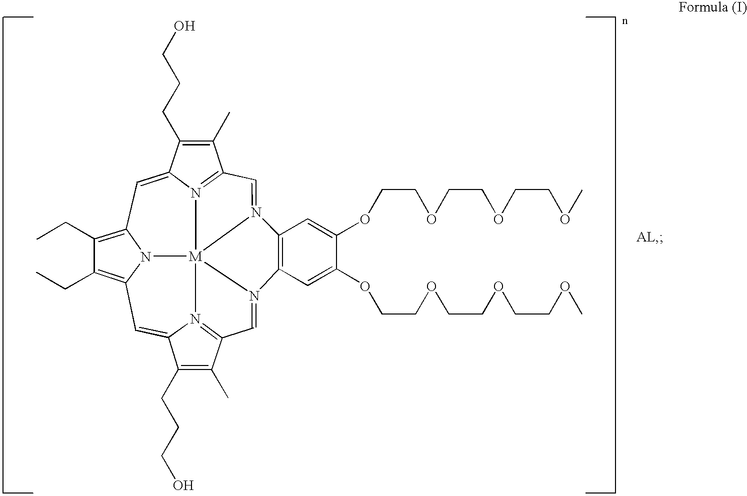
wherein M represents a diamagnetic metal cation selected from Lu(III), Y(III), Cd(II), In(III), and Zn(II). A preferred embodiment provides a process wherein the compound of Formula (I) is motexafin lutetium (MLu, M represents Lu (III)) and wherein the host (including a human patient) is administered: a) from about 0.5 mg/Kg to about 4.0 mg/Kg of MLu; and (b) light fluence in the range of about 20 to about 100 joules/cm.
Pharmaceutical Compositions

The pharmaceutical compositions of a photo-activatable compound (including a compound of Formula (I)) for use in treating vascular inflammation may be administered in either single or multiple doses by any of the accepted modes of administration, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer.

One mode for administration is parental, particularly by injection. The forms in which the compositions described herein may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

At high concentrations, texaphyrins have a tendency to aggregate in aqueous solution, which potentially decreases their solubility. Aggregation may significantly alter the photochemical characteristics of the macrocycles in solution, which is shown by large spectral changes, decrease in extinction coefficient, etc. Addition of a carbohydrate, saccharide, polysaccharide, or polyuronide to the formulation decreases the tendency of the texaphyrin to aggregate, thus increasing the solubility of the texaphyrin in aqueous media. Examples of such agents are sugars, including mannitol, dextrose or glucose. In one embodiment, mannitol is used at concentrations of about 2-8% concentration, including about 5% concentration. These aqueous solutions are suitable for intravenous, intramuscular, subcutaneous and intraperitoneal administration.

Prolonged absorption of the injectable compositions can be brought about by the use in the compositions of agents delaying absorption, for example, aluminum monostearate and gelatin. These particular aqueous solutions are suitable for intra-arterial, intravenous, intramuscular, subcutaneous and intraperitoneal administration.

Sterile injectable solutions are prepared by incorporating a photo-activatable compound (including a compound of Formula (I)) in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

A photo-activatable compound (including a compound of Formula (I)) may be impregnated into a stent by diffusion, for example, or coated onto the stent such as in a gel form, for example, using procedures known to one of skill in the art in light of the present disclosure.

Oral administration is another route for administration, including via capsule or enteric coated tablets, or the like, which prevent degradation of the therapeutic agents described herein in the stomach. In making the pharmaceutical compositions that include a photo-activatable compound (including a compound of Formula (I)), the photo-activatable compound (including a compound of Formula (I)) is usually diluted by an excipient and/or enclosed within such a carrier that can be in the form of a capsule, sachet, paper or other container. When the excipient serves as a diluent, in can be a solid, semi-solid, or liquid material (as above), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents.

The compositions described herein can be formulated so as to provide quick, sustained or delayed release of the active ingredient after administration to the patient by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902514; and 5,616,345. Another formulation for use in the methods described herein employs transdermal delivery devices (“patches”). Such transdermal patches may be used to provide continuous or discontinuous infusion of the therapeutic agents described herein in controlled amounts. Examples of the construction and use of transdermal patches for the delivery of pharmaceutical agents is described in U.S. Pat. Nos. 5,023,252, 4,992,445 and 5,001,139. Such patches may be constructed for continuous, pulsatile, or on demand delivery of pharmaceutical agents.

The compositions may be formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient (e.g., a tablet, capsule, ampoule). For oral administration, each dosage unit contains from 10 mg to 2 g of a compound Formula (I), and for parenteral administration, from 10 to 700 mg of a compound of Formula (I), preferably about 350 mg. The amount of a photo-activatable compound (including a compound of Formula (I)) actually administered should be determined by a physician, in the light of the relevant circumstances, including the condition to be treated, the chosen route of administration, the actual compound administered and its relative activity, the age, weight, and response of the individual patient, the severity of the patient's symptoms, and the like.

For preparing solid compositions such as tablets, the photo-activatable agent, including a compound of Formula (I), is mixed with a pharmaceutical excipient to form a solid preformulation composition. When referring to these preformulation compositions as homogeneous, it is meant that the active ingredient is dispersed evenly throughout the composition so that the composition may be readily subdivided into equally effective unit dosage forms such as tablets, pills and capsules.

The tablets or pills of the present invention may be coated or otherwise compounded to provide a dosage form affording the advantage of prolonged action, or to protect from the acid conditions of the stomach. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions for inhalation or insufflation are administered by the oral or nasal respiratory route for local or systemic effect. Compositions in pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions for inhalation or insufflation may be administered, orally or nasally, from devices that deliver the formulation in an appropriate manner.

Administration for Photodynamic Therapy

By way of example, a photo-activatable compound, such as lutetium texaphyrin may be administered in solution, optionally in 5% mannitol USP. Dosages of about 1.0-2.0 mg/kg to about 8.0-11.0 mg/kg, preferably 3.0 mg/kg, are employed, although in some cases a maximum tolerated dose may be higher, for example about 5 mg/kg. The texaphyrin is administered by intravenous injection, followed by a waiting period of from as short a time as several minutes or about 3 hours to as long as about 72 or 96 hours (depending on the treatment being effected) to facilitate intracellular uptake and clearance from the plasma and extracellular matrix prior to the administration of photoirradiation. Lower dosage ranges may be used for intra-arterial injection or for impregnated stents.

The co-administration of an anti-emetic, a sedative (e.g., benzodiazapenes) and narcotics/analgesics are sometimes recommended prior to light treatment along with topical administration of a local anesthetic, for example Emla cream (lidocaine, 2.5% and prilocalne, 2.5%) under an occlusive dressing. Other intradermal, subcutaneous and topical anesthetics may also be employed as necessary to reduce discomfort. Subsequent treatments can be provided after approximately 21 days.

The optimum length of time following administration of Formula (I) until light treatment can vary depending on the mode of administration, the form of administration, and the type of target tissue. Typically, compounds of Formula (I) persist for a period of minutes to hours, depending on the compound of Formula (I), the formulation, the dose, the infusion rate, as well as the type of tissue and tissue size. The light source for the photodynamic therapy may be a laser, a light-emitting diode, or filtered light from, for example, a xenon lamp; and the light may be administered topically, endoscopically, or interstitially (via, e.g., a fiber optic probe), or intraarterially.

Packaged Product for the Treatment of Vascular Inflammation

Compounds of Formula (I) for use in treating vascular inflammation may be prepared for packaging in different forms, including by way of example only, as a solution or powder. Depending on the form of Formula (I), an appropriate container suitable to hold Formula (I) may be used. Also dependent upon the container chosen, sealing the container and adjusting the environment inside the container for packing will be done. Optional steps may involve adding extra materials either to the container or along with the container for packaging, by way of example only includes a bottle top, desiccants, tamper-proof seal, plastic wrap and the like. Finally the sealed container containing Formula (I) is packaged within an appropriate outer package.

Types of Outer Packaging

In one embodiment, the outer packaging is a paper box. In one embodiment, the outer packaging protects the container with seal and contents (a solution of Formula (I)) from light. In further or alternative embodiments, the outer packaging protects the container with seal along with an aluminum seal protector and its contents of Formula (I) from sunlight, ultraviolet light, contaminants, degradation, impurities, other solutions and spillage. The outer packaging will not significantly absorb, react with, or otherwise adversely affect the Formula (I) drug or other excipients or components used in intravenous delivery during storage of the drug prior to its use. The outer packaging may be in any shape or form which protects container with seal and its contents of Formula (I), including, by way of example only a paper box, a cardboard box, a carton, a plastic bag, a fabric case, a metal receptacle, a wooden bin or the like.

The qualification standards for packaging a vial or sealed container within an outer packaging depends on the type of vial or sealed container and/or the type of outer packaging. In one embodiment, a combination of a sealed glass vial and paper box is used as protection material for packaging of Formula (I). Such a combination provides maximum protection from light degradation as well as oxygen degradation. Such varied combinations of packaging also provide a protective environment for solutions containing Formula (I) from outside temperatures ranging from about 0-3° C., about 2.5-4.5° C., about 3.5-5.5° C., about 4.5-7.5° C. and about 5.0-8.5° C. In one embodiment, these storage-stabilized packaged formulations are stored at room temperature or in a standard refrigerator or at temperatures from about 2 to 8° C. or about 2 to 5° C.

Further or alternative embodiments, by way of example only, include a combination of a syringe sealed in plastic with a cardboard box, a combination of a syringe sealed in plastic with an outer nontransparent paper lining, a combination of a glass bottle sealed in plastic with a cardboard box, a combination of a plastic bottle with a cardboard box, a combination of a plastic bottle sealed in plastic with a cardboard box, a combination of a glass bottle encased in a Styrofoam case within a cardboard box, a combination of a syringe encased in a Styrofoam case within a cardboard box, and the like. The qualification standards for other such combinations of sealed containers and outer packaging differ because of the different materials used in the container and outer packaging. However, any combination should provide protection from contamination, such as the crystallization or degradation, of the drug, and from other environmental factors, during storage of the system prior to its use. Further, the outer packaging may contain a dessicant or an oxygen-absorbing material.

Types of Containers

In one embodiment, the vial or container that contains the compound of Formula (I) has a seal and fits into an outer packaging. The container aids to protect its contents of Formula (I) from contaminants, degradation, impurities, other solutions and/or spillage. The container forms a protective environment to house Formula (I) so as to slow the effects of degradation of Formula (I). Further alternative embodiments of different container types include, by way of example only, a high density polyethylene container, a plastic bottle, a syringe, a “drip bag,” a pre-filled syringe, an intravenous bag, and the like. In further or alternative embodiments, the container contains a compound of Formula (I) in solution—including a concentrated solution that can be diluted down to a desired concentration or at a concentration ready for administration to the patient. Alternatively, the container can contain a solid dosage form of a compound of Formula (I), wherein the solid dosage form can be dissolved in an appropriate solution to create a formulation having a desired concentration. The solid dosage form can include a powder, including a lyophilized powder; semi-crystalline material; crystalline material; grains; granules and the like. Alternatively, the container can contain a semi-solid dosage form of a compound of Formula (I), including a gel or jelly, wherein the semi-solid dosage form can be dissolved in an appropriate solution to create a formulation having a desired concentration. Thus, in any of the container embodiments described herein, the compound of Formula (I) can be in the form of a solid, semi-solid, or solution, and further may be either ready to use (i.e., administer to a patient), or available for formulation to a desired pharmaceutical dosage form, including an intravenously-acceptable formulation.

Qualification Standards for Containers

The qualification standards for a vial or sealed container varies depending on the type of vial or sealed container used and which form of Formula (I) is used. By way of example only, a sealed syringe housing a powder form of Formula (I) or a sealed bottle housing a powder form of Formula (I) may withstand higher temperatures than a sealed syringe housing a liquid form of Formula (I) or a sealed bottle housing a liquid form of Formula (I) which may lead to a higher rate of degradation of the drug. In one embodiment, the container housing the drug is in an oxygen depleted environment which is sealed and substantially airtight. However, any combination should provide protection from contamination, such as the crystallization or degradation, of the drug, and from other environmental factors, during storage of the system prior to its use.

In one embodiment, the liquid form of Formula (I) is housed in a container with a minimal amount of headspace for storage. The headspace may contain at least about 90% nitrogen gas, or at least about 95% nitrogen gas and occupy either less than about 12% or less than about 7% of the volume of the sealed container. In still a further embodiment, the liquid form of Formula (I) is flushed with nitrogen inside the container. In a further embodiment, a non-oxygen gas (including nitrogen, argon, neon or combinations thereof) is flushed into the empty container followed by the solution of Formula (I); alternatively, the solution of Formula (1) partially fills the container and the remaining head space is flushed with a non-oxygen gas.

In further or alternative embodiments, a protective cap may accompany the bottle seal or syringe tip seal. The protective cap may prevent unintentional damage to the bottle or syringe tip seal before use. In another embodiment, the protective cap may be child-resistant to prevent unintentional opening by a minor before use. In still further or alternative embodiments, a plastic bag, a foil wrapped container or other such materials may seal the vial and/or sealed container within the outer packaging. The plastic bag or foil wrapped container may provide another protective layer against light, contaminants, degradation, impurities, other solutions and spillage.

Forms of Packaged Formula (I)

Any of the pharmaceutical compositions and formulations described herein may be packaged as described herein. One embodiment described herein is a packaged product of Formula (I) for intravenous drug use to a human subject wherein the packaging will not significantly absorb, react with, or otherwise adversely affect the drug or other excipients or components used in intravenous delivery during storage of the system prior to its use. In a further embodiment described herein are packaged products of Formula (I) for intravenous delivery, comprising a high-purity texaphyrin metal complex of Formula (I). The foregoing and other objectives are achieved by providing light protective materials and a substantially deoxygenated environment to prevent degradation to Formula (I) prior to use. Such light protective materials include an outer packaging that is opaque and an inner package that comprises a transparent, non-tinted material, such as glass. The packaging of Formula (I) for intravenous use is dependent on the form of the drug. In one embodiment, Formula (I) may be packaged in liquid form. In another embodiment, Formula (I) may be packaged in powder form with reconstituting solution.

Suitable storage-stabilized formulations of Formula (I) include a solution of Formula (I) in water and acetic acid. In one embodiment, the storage-stabilized formulation should have a pH of 5.4. In other embodiments, the storage-stabilized formulation should have a pH between about 4.5-5.5, about 5.0-5.9 or about 4.9-5.9. In another embodiment the concentration of Formula (I) in the storage-stabilized formulation is between 2.5 mg/mL and about 3.0 mg/mL; in a further embodiment the concentration of Formula (1) is about 2.5 mg/mL.

In further or alternative embodiments, storage-stabilized formulation contains an isotonic agent, which can include electrolytes and/or non-electrolytes. Non-limiting examples of electrolytes includes sodium chloride, potassium chloride, dibasic sodium phosphate, sodium gluconate and combinations thereof. Non-limiting examples of non-electrolytes includes saccharides and polyhydric alcohols; further examples include mannitol, sorbitol, glucose, dextrose, glycerol, xylitol, fructose, maltose, mannose, glycerin, propylene glycol, and combinations thereof. In still further embodiments, the storage-stabilized formulation comprises a buffer, an anti-crystallizing agent, and/or a preservative. Buffering agents aid in stabilizing pH. Anti-crystallizing agents aid in stabilizing the concentration of the solution. Preservatives aid in preventing the growth of micro-organisms, and include by way of example only, methyl paraben, propyl paraben, benzyl alcohol, sodium hypochlorite, phenoxy ethanol and/or propylene glycol. In one, the storage-stabilized formulation does not contain an oxidizing agent other than Formula (I) and oxygen. Oxidizing agents promote degradation of the compound of Formula (I).

General Packaging Specifications

The packaging system may be prepared by loading the product package contents (i.e., Formula (I), bottle, syringe, plastic bag, desiccant, cardboard box) by means of any suitable or conventional manufacturing operation and sealing process. The sealing process may include gas flushing or evacuation of oxygen from the container.

The degradation of solutions comprising Formula (I) can be measured by the levels of free Lu³⁺. In one embodiment, accumulation of less than 30 ppm of free metal ion, including Lu⁺³, within the packaged product is desired. In another embodiment, the accumulation of free metal ion, including Lu⁺³, within the packaged product should not exceed 30 ppm for at least about 1 year. In yet another embodiment, the accumulation of free metal ion, including Lu⁺³, within the packaged product should not exceed 30 ppm for at least about 3 years. Whether Formula (I) is packaged as a solution or powder form for reconstitution before use, measurement of Lu⁺³ levels can be an indication of degradation or spoilage.

In one embodiment, Formula (I) may be packaged in powder form with reconstituting solution. Reconstitution is achieved by admixing the Formula (I) powder with a solution comprising, e.g., water, acetic acid and mannitol, using amounts and concentrations as described for the Formula (I) solutions described herein. The term “powder” is used to generically describe any solid form of Formula (I) in a particulate form, including crystalline forms and non-crystalline forms, or grains, beads, chunks, fine powders, coarse powder or other particulate forms.

In one embodiment, the container is a non-tinted borosilicate glass vial, USP Type I. The vial can hold a sufficient amount of a solution of Formula (I) to allow reliable administration of 50 mL of such a solution to a patient (which generally means the vial can hold 51-53 mL of solution). Further, such a vial has a suitable head space and an opening of 20 mm. Further, the seal for the container is a one piece elastomeric bottle stopper composed of butyl rubber which forms a tight seal onto a glass bottle container housing Formula (I). In this embodiment, the stopper is a 20 mm flange type constructed from 4405/50 gray butyl rubber and laminated at the product contact area with a Teflon® film. Teflon) is fluorinated ethylene-propylene (FEP) applied as a film to the face of the stopper. The seal diameter is 20 mm and the seal is constructed of aluminum with a violet colored plastic Flip-Off® button.

Each vial is packaged in an individual vial carton to afford protection from light. The cartons are made from 0.024 inch thick solid bleached sulfate paper and are coated on the outside. The base color of the carton exterior is bright white and cartons are imprinted with labeling text. The cartons are provided flat and are folded during packaging operations. One vial is placed per carton and the carton is folded or glued closed. The final dimensions of the folded and closed carton are 1¾ inches wide×1¾ inches deep×3¼ inches high.

Combination Therapies

Because of the inflammatory aspect of vascular inflammation, compounds of Formula (I) or other photo-activatable compounds may be administered to a patient in conjunction with anti-inflammatory agents, including by way of example only indomethacin, acetylsalicylic acid (aspirin), ibuprofen, sulindac, phenylbutazone, naproxen, diclofenac, celecoxib, resveratrol, CAY 10404 and curcumin. When administered in a combination, the compound of Formula (I) or other photo-activatable compounds can be administered before, simultaneously and/or after the anti-inflammatory agent. The time between administration of a compound of Formula (I) or other photo-activatable compounds and administration of an anti-inflammatory agent can be between 0 seconds (i.e., the two agents are administered simultaneously) to 1 week. When administered simultaneously, the two agents may be given in the same pharmaceutical dose or in separate pharmaceuticals doses.

A photo-activatable compound of Formula (I) may also be administered to a patient in conjunction with a zinc reagent, including by way of example only zinc acetate, zinc chloride, zinc citrate, zinc lactate, and zinc complex of 1-hydroxypyridine-2-thione. For further details on the administration of compounds of Formula (I) in conjunction with zinc reagents see International Application No. PCT/US/2005/017812, the disclosure of which is incorporated by reference in its entirety. When administered in a combination, the compound of Formula (I) or other photo-activatable compounds can be administered before, simultaneously and/or after the zinc reagent. The time between administration of a compound of Formula (I) or other photo-activatable compounds and administration of a zinc reagent can be between 0 seconds (i.e., the two agents are administered simultaneously) to 1 week. When administered simultaneously, the two agents may be given in the same pharmaceutical dose or in separate pharmaceuticals doses.

A photo-activatable compound of Formula (I) may also be administered to a patient in conjunction with a cellular metabolite that increases the reactive oxygen species production in the atheroma, vulnerable plaque, or site of inflammation in the blood vessel. Such a cellular metabolite includes, by way of example only ascorbate, NADPH, NADH, FADH₂ and reduced glutathione. For further details on the administration of compounds of Formula (I) in conjunction with such cellular metabolites see U.S. Pat. No. 6,825,186, the disclosure of which is incorporated by reference in its entirety. When administered in a combination, the compound of Formula (I) or other photo-activatable compounds can be administered before, simultaneously and/or after such cellular metabolites. The time between administration of a compound of Formula (I) or other photo-activatable compounds and administration of such cellular metabolites can be between 0 seconds (i.e., the two agents are administered simultaneously) to 1 week. When administered simultaneously, the two agents may be given in the same pharmaceutical dose or in separate pharmaceuticals doses.

### EXPERIMENTAL

Synthesis

Compounds of Formula (I) can be synthesized by procedures outlined in U.S. Pat. Nos. 4,935,498, 5,252,720, 5,801,229, 5,451,576, 5,569,759, and 6,638,924, and U.S. patent application Ser. No. 11/235,475 filed on Sep. 26, 2005, the disclosures of which are incorporated by reference in their entirety. Compounds of Formula (I) can be formulated into an intravenously-acceptable pharmaceutical formulation, and stored as such a formulation, as described in U.S. Pat. Nos. 6,919,327 and 6,638,924, and U.S. patent application Ser. No. 11/241,549 filed on Sep. 30, 2005, the disclosures of which are incorporated by reference in their entirety

Methods

Thirty 8 months old male Watanabe Heritable Hyperlipidemic (WHHL) rabbits were randomized after 3 weeks of 1% cholesterol feeding to either control or MLu groups (10 mg/kg, iv via ear vein). Intravascular illumination was performed 24 hours post-MLu using a fiber with a 30 mm active diffuser tip to deliver escalating fluences (30-175 J/cmf, 732 nm) for 400 s (upper) and 900 s (lower thoracic/abdominal aorta); control received no light. Treatment regimens are shown in the table below. Rabbits were then switched to a normal diet immediately after phototherapy and sacrificed 3 weeks later. Aortas were harvested and examined for plaque (I/M ratios), Mac (RAM11), smooth muscle cell (myosin) and collagen (Picrosirius Red) changes.

TABLE
Light regimens tested using a 30 mm diffuser

| | | Fluence rate | Fluence rate |
|---|---|---|---|
| | Total fluences | (mW/cm^{f}) | (mW/cm^{f}) |
| Group | (J/cm^{f}) | Top site (400 s) | Bottom site (900 s) |
| 1 | N/A (control) | N/A | N/A |
| 2 | 30 | 75 | 33 |
| 3 | 75 | 188 | 83 |
| 4 | 125 | 313 | 139 |
| 5 | 175 | 438 | 194 |

It is understood that the compositions, methods, strategies, processes, therapies, and techniques described herein (a) can be used to stabilize plaque on or within arteries by using a photo-activatable compound, including Texaphyrins, and light fluence in the range of about 20 joules/cm to about 400 joules/cm; (b) can be used to treat diffuse inflammation along extended segments of a blood vessel; (c) can also be used to treat extended segments of a blood vessel wherein the inflammation is non-continuous and wherein treating the whole segment does not adversely affect the healthy or the non-inflamed portion of the blood vessel; and (d) can be used to treat inflammation that may not be detected yet is present near detectable inflammation within a blood vessel.

While embodiments have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed in practicing the invention. It is intended that the following claims define the scope of the invention and that methods and structures within the scope of these claims and their equivalents be covered thereby.

We claim:

1. A process for treating diffuse regional inflammation in blood vessels comprising administering to a human patient in need of such treatment a photo-activatable compound and light delivered endovascularly at a fluence in the range of about 10 to about 400 Joules/cm to segments of the blood vessels.

2. The process of claim 1 wherein the light activatable compound has the structure of Formula (I): 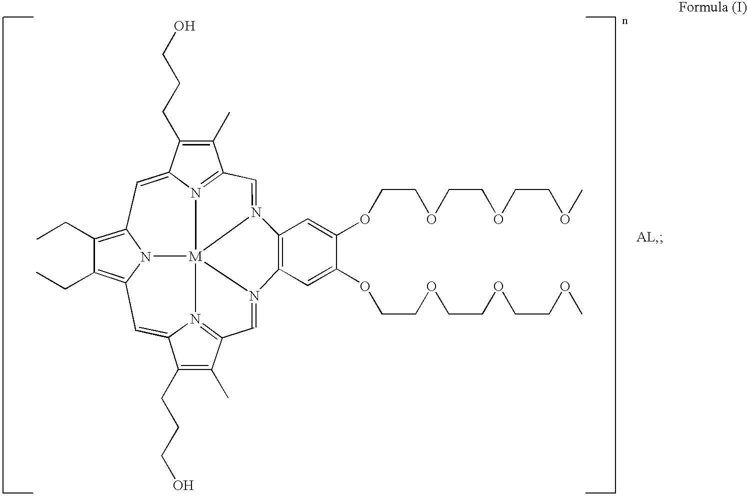
wherein M is selected from Lu(III), Y(III), Cd(II), In(III), and Zn(II); wherein n is 1 or 2; and AL is an apical ligand derived from the group consisting of hydrochloric acid, nitric acid, acetic acid, gluconic acid, glucoronic acid, cholic acid, deoxycholic acid, methylphosphonic acid, phenylphosphonic acid, phosphoric acid, formic acid, propionic acid, butyric acid, pentanoic acid, 3,6,9-trioxodecanoic acid, 3,6-dioxoheptanoic acid, 2,5-dioxoheptanoic acid, methylvaleric acid, glycolic acid, pyruvic acid, oxalic acid, malic acid, malonic acid, succinic acid, maleic acid, fumaric acid, tartaric acid, citric acid, methanesulfonic acid, ethanesulfonic acid, benzoic acid, salicylic acid, 3-fluorobenzoic acid, 4-aminobenzoic acid, cinnamic acid, mandelic acid, and p-toluene-sulfonic acid.

3. The process of claim 2 wherein M is Lu(III), and n is 2.

4. The process of claim 3 wherein the human patient is administered:
(a) from about 0.5 mg/Kg to about 4.0 mg/Kg of the compound of Formula (I); and
(b) light fluence in the range of about 20 to about 100 joules/cm of the segments of the blood vessels.

5. A process for treating vulnerable plaque in segments of a coronary artery, arterial graft or vein graft in a human patient needing such treatment, such process comprising administering to the human patient a photo-activatable compound and administering light endovascularly at a fluence in the range of about 10 to about 400 joules/cm.

6. The process of claim 5 wherein the light activatable compound has the structure of Formula (I): 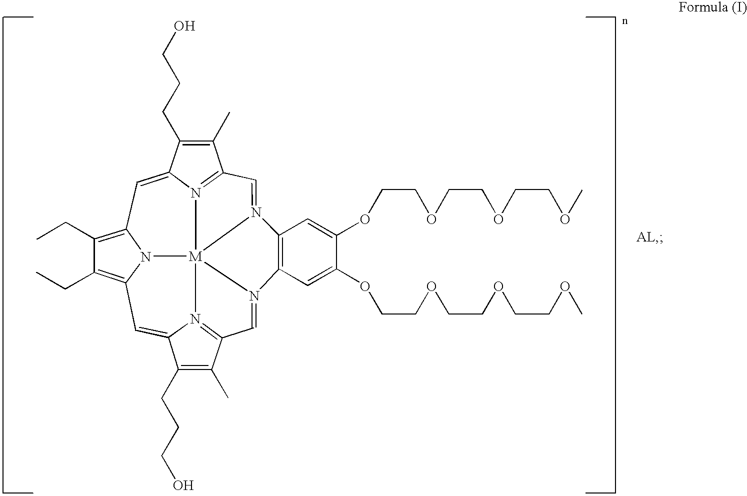
wherein M is selected from Lu(III), Y(III), Cd(II), In(III), and Zn(II); wherein n is 1 or 2; and AL is an apical ligand derived from the group consisting of hydrochloric acid, nitric acid, acetic acid, gluconic acid, glucoronic acid, cholic acid, deoxycholic acid, methylphosphonic acid, phenylphosphonic acid, phosphoric acid, formic acid, propionic acid, butyric acid, pentanoic acid, 3,6,9-trioxodecanoic acid, 3,6-dioxoheptanoic acid, 2,5-dioxoheptanoic acid, methylvaleric acid, glycolic acid, pyruvic acid, oxalic acid, malic acid, malonic acid, succinic acid, maleic acid, fumaric acid, tartaric acid, citric acid, methanesulfonic acid, ethanesulfonic acid, benzoic acid, salicylic acid, 3-fluorobenzoic acid, 4-aminobenzoic acid, cinnamic acid, mandelic acid, and p-toluene-sulfonic acid.

7. The process of claim 6 wherein M is Lu(III), and n is 2.

8. The process of claim 7 wherein the human patient is administered:
(a) from about 0.5 mg/Kg to about 4.0 mg/Kg of the compound of Formula (I); and
(b) light fluence in the range of about 20 to about 100 joules/cm.

9. The process of claim 6, wherein the vulnerable plaque is composed of inflammatory cells and lipids.

10. A process for treating a segment of a blood vessel containing diffuse involvement by atheromatous plaque disease, the process comprising administering to a human patient in need of such a treatment a photo-activatable compound and light delivered endovascularly at fluence in the range of from about 10 joules/cm to about 400 joules/cm along a segment of the blood vessel.

11. The process of claim 10 wherein the light activatable compound has the structure of Formula (I): 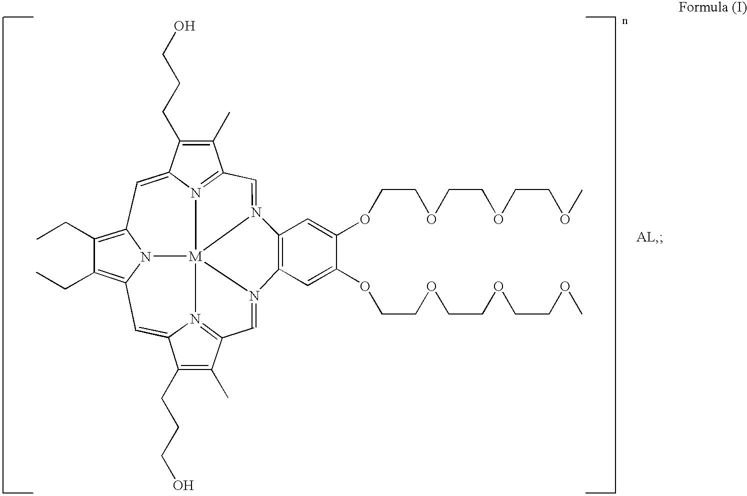
wherein M is selected from Lu(III), Y(III), Cd(II), In(III), and Zn(II); wherein n is 1 or 2; and AL is an apical ligand derived from the group consisting of hydrochloric acid, nitric acid, acetic acid, gluconic acid, glucoronic acid, cholic acid, deoxycholic acid, methylphosphonic acid, phenylphosphonic acid, phosphoric acid, formic acid, propionic acid, butyric acid, pentanoic acid, 3,6,9-trioxodecanoic acid, 3,6-dioxoheptanoic acid, 2,5-dioxoheptanoic acid, methylvaleric acid, glycolic acid, pyruvic acid, oxalic acid, malic acid, malonic acid, succinic acid, maleic acid, fumaric acid, tartaric acid, citric acid, methanesulfonic acid, ethanesulfonic acid, benzoic acid, salicylic acid, 3-fluorobenzoic acid, 4-aminobenzoic acid, cinnamic acid, mandelic acid, and p-toluene-sulfonic acid.

12. The process of claim 11 wherein M is Lu(III) and n is 2.

13. The process of claim 12 wherein the human patient is administered:
(a) from about 0.5 mg/Kg to about 4.0 mg/Kg of the compound of Formula (I); and
(b) light fluence in the range of about 20 to about 100 joules/cm of the segments of the blood vessels.

14. A process of treating a non-continuous inflammatory condition involving a blood vessel, the process comprising administering to a human patient in need of such a treatment a photo-activatable compound and light delivered endovascularly at fluence in the range of from about 10 joules/cm to about 400 joules/cm along a segment of the blood vessel containing the non-continuous inflammation.

15. The process of claim 14 wherein the light activatable compound has the structure of Formula (I): 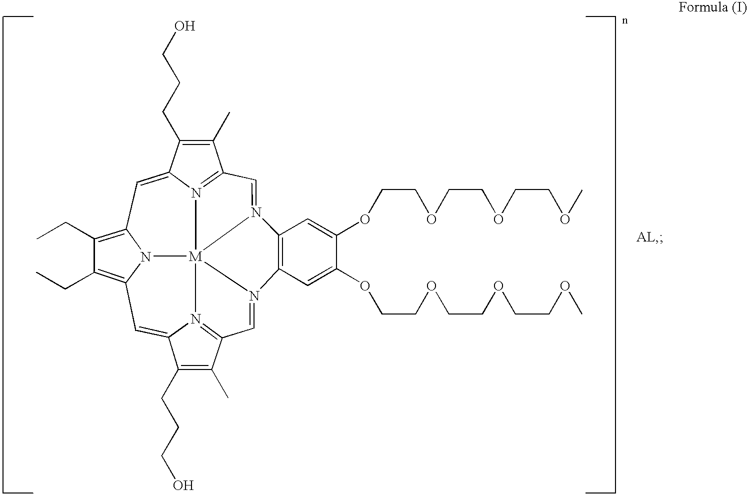
wherein M is selected from Lu(III), Y(III), Cd(II), In(III), and Zn(II); wherein n is 1 or 2; and AL is an apical ligand derived from the group consisting of hydrochloric acid, nitric acid, acetic acid, gluconic acid, glucoronic acid, cholic acid, deoxycholic acid, methylphosphonic acid, phenylphosphonic acid, phosphoric acid, formic acid, propionic acid, butyric acid, pentanoic acid, 3,6,9-trioxodecanoic acid, 3,6-dioxoheptanoic acid, 2,5-dioxoheptanoic acid, methylvaleric acid, glycolic acid, pyruvic acid, oxalic acid, malic acid, malonic acid, succinic acid, maleic acid, fumaric acid, tartaric acid, citric acid, methanesulfonic acid, ethanesulfonic acid, benzoic acid, salicylic acid, 3-fluorobenzoic acid, 4-aminobenzoic acid, cinnamic acid, mandelic acid, and p-toluene-sulfonic acid.

16. The process of claim 15 wherein M is Lu(III) and n is 2.

17. The process of claim 16 wherein the human patient is administered:
(a) from about 0.5 mg/Kg to about 4.0 mg/Kg of the compound of Formula (I); and
(b) light fluence in the range of about 20 to about 100 joules/cm of the segments of the blood vessels.

18. A process of treating inflammation involving multiple blood vessels, the process comprising administering to a human patient in need of such a treatment a photo-activatable compound and light delivered endovascularly at fluence in the range of from about 10 joules/cm to about 400 joules/cm to inflamed segments of each of the multiple blood vessels containing the inflammation.

19. The process of claim 18 wherein the light activatable compound has the structure of Formula (I): 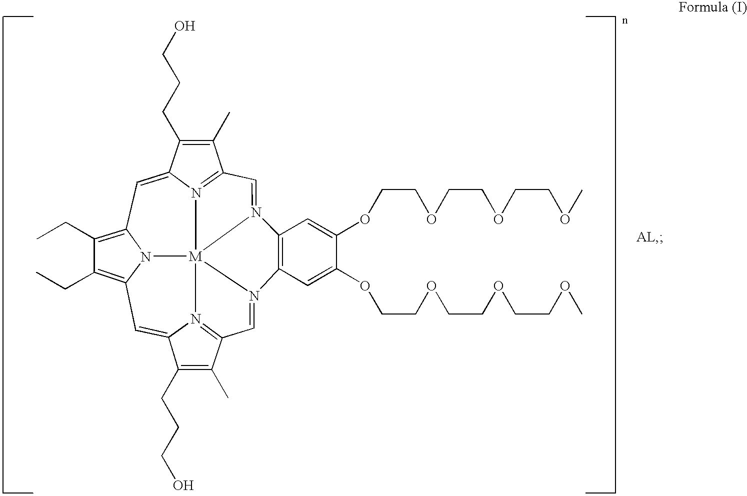
wherein M is selected from Lu(III), Y(III), Cd(II), In(III), and Zn(II); wherein n is 1 or 2; and AL is an apical ligand derived from the group consisting of hydrochloric acid, nitric acid, acetic acid, gluconic acid, glucoronic acid, cholic acid, deoxycholic acid, methylphosphonic acid, phenylphosphonic acid, phosphoric acid, formic acid, propionic acid, butyric acid, pentanoic acid, 3,6,9-trioxodecanoic acid, 3,6-dioxoheptanoic acid, 2,5-dioxoheptanoic acid, methylvaleric acid, glycolic acid, pyruvic acid, oxalic acid, malic acid, malonic acid, succinic acid, maleic acid, fumaric acid, tartaric acid, citric acid, methanesulfonic acid, ethanesulfonic acid, benzoic acid, salicylic acid, 3-fluorobenzoic acid, 4-aminobenzoic acid, cinnamic acid, mandelic acid, and p-toluene-sulfonic acid.

20. The process of claim 19 wherein M is Lu(III) and n is 2.

21. The process of claim 20 wherein the human patient is administered:
(a) from about 0.5 mg/Kg to about 4.0 mg/Kg of the compound of Formula (I); and
(b) light fluence in the range of about 20 to about 100 joules/cm of the segments of the blood vessels.

22. A process of treating non-continuous inflammation in multiple blood vessels, the process comprising administering to a human patient in need of such a treatment a photo-activatable compound and light delivered endovascularly at fluence in the range of from about 10 joules/cm to about 400 joules/cm along the entire inflamed segments of the multiple segments of the blood vessels containing the non-continuous inflammation.

23. The process of claim 22 wherein the light activatable compound has the structure of Formula (I): 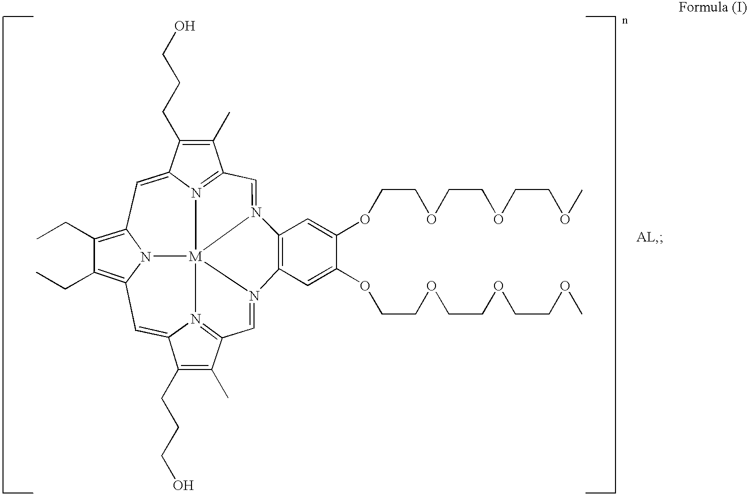
wherein M is selected from Lu(III), Y(III), Cd(II), In(III), and Zn(II); wherein n is 1 or 2; and AL is an apical ligand derived from the group consisting of hydrochloric acid, nitric acid, acetic acid, gluconic acid, glucoronic acid, cholic acid, deoxycholic acid, methylphosphonic acid, phenylphosphonic acid, phosphoric acid, formic acid, propionic acid, butyric acid, pentanoic acid, 3,6,9-trioxodecanoic acid, 3,6-dioxoheptanoic acid, 2,5-dioxoheptanoic acid, methylvaleric acid, glycolic acid, pyruvic acid, oxalic acid, malic acid, malonic acid, succinic acid, maleic acid, fumaric acid, tartaric acid, citric acid, methanesulfonic acid, ethanesulfonic acid, benzoic acid, salicylic acid, 3-fluorobenzoic acid, 4-aminobenzoic acid, cinnamic acid, mandelic acid, and p-toluene-sulfonic acid.

24. The process of claim 23 wherein M is Lu(III) and n is 2.

25. The process of claim 24 wherein the human patient is administered:
(a) from about 0.5 mg/Kg to about 4.0 mg/Kg of the compound of Formula (I); and
(b) light fluence in the range of about 20 to about 100 joules/cm of the segments of the blood vessels.
